(19) United States
(12) Patent Application Publication
(10) Pub. No.: US 2022/0008757 A1
(43) Pub. Date: Jan. 13, 2022

(54) Portable air purifier

(71) Applicant: Thomas Lawlor, Lake Worth, FL (US)

(72) Inventor: Thomas Lawlor, Lake Worth, FL (US)

(21) Appl. No.: 17/369,455

(22) Filed: Jul. 7, 2021

Related U.S. Application Data
(60) Provisional application No. 63/050,451, filed on Jul. 10, 2020.

(51) Int. Cl. A62B 7/10; B01D 46/42; B01D 46/00; A62B 18/00; A62B 18/08; A62B 23/02; A62B 9/00; A41D 13/11

(52) U.S. Cl. CPC A62B 7/10; B01D 46/4227; B01D 46/4245; B01D 46/0049; A62B 18/006; A62B 7/12; A62B 23/02; A62B 9/00; A62B 18/084; A41D 13/1184; B01D 2273/30; A62B 18/086

(57) ABSTRACT

A face mask having a positive air flow by use of an attached portable air purification unit. The device is used to filter and purify inhaled air, making it easier for individuals to comfortably wear a face mask for public health and safety. An outtake filter inhibits contaminated air from being expelled by the mask wearer. The portable air purifier is powered by means of rechargeable battery power using a USB connector, allowing static or dynamic use.

### FEDERALLY SPONSORED RESEARCH OR DEVELOPMENT

Not applicable.

### PRIORITY CLAIM

In accordance with 37 C.F.R. § 1.76, a claim of priority is included in an Application Data Sheet filed concurrently herewith. Accordingly, the present invention claims priority to U.S. Provisional Patent Application No. 63/050,451, entitled “PORTABLE FACE MASK AIR PURIFIER,” filed Jul. 10, 2020. The contents of the above referenced application are incorporated herein by reference.

### FIELD OF THE INVENTION

The present invention relates to air purifiers and, in particular, to a portable air purifier.

### BACKGROUND OF THE INVENTION

As a result of the rapid spread of the novel corona virus (COVID-19), many municipalities, cities and states have instituted requirements for wearing of face coverings or face masks inside public buildings where social distancing is not possible. Concerts, weddings, funerals, schools, colleges, food production, shopping, sporting events, and so forth, have all become areas that need to be addressed to prevent the rapid spread of COVID-19. Face masks are especially important should an individual be actively infected, or possibly be an asymptomatic carrier of the virus. At present, there is a vaccine for the virus, but limited access to the vaccine and related medical treatment. Variants of the virus are occurring, and many individuals are susceptible to other airborne viruses, including the flu and common cold, that can result in serious health conditions. There remains a sizable population that cannot, or will not, take the vaccine even if available. For instance, individuals with immune deficiencies, cancer patients, individuals taking immune suppressing medications, individuals with various religious beliefs, and so forth. Further, there are times wherein airborne contaminants can result in serious health issues. For instance, Arizona is prone to dust clouds; California is prone to smog; Florida is prone to pollen. African dust can affect the entire south eastern section of the U.S. In the western sections of the country, wildfires affect nearly everyone from Colorado to southern California and Washington State. Depending on the individual, airborne particulates can result in a serious health issue.

Unfortunately, the wearing of a face mask can be uncomfortable for many individuals, particularly those with breathing issues, such as COPD or asthma, wherein wearing a mask further inhibits the ability to breathe. Face masks require an individual to draw air through a filter, which can be increasingly difficult with the better quality mask materials, such as the N95. Further, an individual must expel air through the mask, which requires an effort, especially if the filter becomes saturated with moisture, dust, pollen and so forth. The N95 medical grade and KN95 non-medical grade masks are considered effective filters to wear based upon the filtering capabilities. However, the breathability of a conventional mask can be problematic at first, and can only deteriorate with use.

It is critical to the health of the public that certain individuals wear a mask. For instance, cooks preparing food in a restaurant, grocery store checkouts, airline check-ins, and so forth. For these individuals, the prospect of wearing a mask for 8 hours a day can be most difficult. If an individual is labored in breathing, it is only natural that the individual will remove the mask, or disable its effectiveness by moving the mask down so they may breathe through their nose.

The KN95 mask is recognized as one of the most effective non-medical filtering masks. The KN95 blocks 95% of the particles 0.3 microns or larger in size. The quality of the filter comes with a disadvantage, in that the filter inhibits normal breathing, as air is both drawn through the filter and then expelled through the filter. If used in a restaurant kitchen, the filter can become more restrictive as the filter traps airborne cooking byproducts. As the filter clogs, the user is left with the difficult task of forcing air through the filter or changing of the filter. Humidity in the air can bind particles caught by the filter, causing further clogging of the filter. HEPA filters capture 99.7% of particles 0.3 microns or smaller in size, but can clog even quicker than the N95. Wearing a face mask having a HEPA filter grade is most difficult, as it requires too much effort to draw and expel air through the material.

Failure to properly exhale through a mask can be harmful to the individual. An individual exhales carbon dioxide, which is a by-product of the process of cell respiration. If the individual does not expel the carbon dioxide from the mask, a risk of inhaling the carbon dioxide occurs, which can reduce or stop the amount of oxygen reaching the brain.

U.S. Pat. No. 5,165,395 discloses a germicidal mask system to kill germs, viruses, or other pathogens which are located in the air to be breathed by the user. Before entering the mask, the air is exposed to ultra-violet radiation, killing the undesirable germs, viruses, and other pathogens.

U.S. Pat. No. 9,737,895 discloses a portable rechargeable personal ionic air purifier that energizes a personal airspace and cleans particulate pollutants therefrom. The ionization means includes an ion emitter, and the ionization activation means includes means for providing a pulse train of predetermined frequency and duty cycle. The ion emitter may be removably mounted to a portable housing and/or to a facemask or other objects in the personal airspace via an elongated flexible cable.

U.S. Pat. No. 7,118,608 discloses a self-powered, personally wearable air purifier that includes a sequence of packed bed filter media packets having filter media therein. A HEPA filter is included for the removal of particulate matter that will have adhered thereto many of such pollutants. Shoulder straps and a chest strap are provided for wearing the apparatus on a user's back; or other means, such as a shoulder pouch, can also be employed.

U.S. Pat. No. 10,821,255 discloses a portable air purifier using ionization electrodes configured to provide an electrochemical treatment of the water inside a water absorbing substance to provide forced purified air.

What is lacking in the art is a low cost apparatus that provides portable fan based air purification using a high quality filter, such as an N95 or HEPA filter, coupled to a face mask, wherein the device purifies the air to be inhaled and assists in the expelling of exhaled air by use of a positive air flow.

### SUMMARY OF THE INVENTION

Disclosed is a personal portable air purifier tethered to a face mask for providing a positive supply of filtered air to an individual using a battery supply. The device has an intake filter used to prevent the inhalation of viruses, pollen and the like, and an outtake filter to inhibit unfiltered exhaled air.

An objective of the instant invention is to provide a positive flow portable air purifier having a air handler of a size and weight that is attachable to an individual's waist by securing to an individual's belt.

Another objective of the invention is to provide a portable air purifier wherein high quality filters, such as a HEPA filter and N95 filter material, can be employed by supplementing air movement to allow extended use of the filters.

A further objective of the instant invention is to provide a portable rechargeable power source that employs a USB connection, allowing recharging from any USB power source, personal PC, automobile, airplane, airport, and so forth.

Yet another objective of the instant invention is to provide a face mask assembly having a filter for incoming air and a filter for expelled air providing full face visibility.

Another objective is to provide a portable air purifier that can operate at least 8 hours of runtime on a single 5000 mAh battery.

Still another objective of the invention is to provide a form fitting face mask that allows an individual to comfortably wear a mask for filtering of air by use of positive air flow, thereby eliminating the need to touch the filter for adjustment.

Another objective of the invention is to provide a personal air purifier that provides positive air flow to assure an individual receives filtered air without effort, and provides positive flow to assure carbon dioxide expulsion.

Yet another objective of the invention is to provide a positive air flow face mask to avoid fogging of glasses, as commonly found on ambient masks.

Yet still another objective of the invention is to provide a portable air purifier that can be used in a static or dynamic configuration.

Still another objective of the invention is to provide a personal air purifier that is lightweight, about 1.2 lbs., and can be easily attached to a waist belt for ease of portability.

Still another objective of the invention is to teach the use of a face mask wherein the filter can be temporarily removed to allow an individual to consumer food or beverage while still being supplied with filtered air.

Another objective of the invention is to provide a personal air purifier having a face mask and intake housing that can be mass produced through injection molding, or 3D printing.

Other objectives and advantages of this invention will become apparent from the following description, taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### BRIEF DESCRIPTION OF THE DRAWINGS

FIG. 1 is a perspective environmental view of the instant invention worn by an individual;

FIG. 2 is a perspective view of the air handler;

FIG. 3 is a side view thereof;

FIG. 4 is an exploded view thereof;

FIG. 5 is a front view of a face mask filter;

FIG. 6 is a perspective view of a face mask support;

FIG. 7 is a perspective view of an alternative embodiment face mask assembly; and

FIG. 8 is an exploded view of FIG. 7.

### DESCRIPTION OF THE PREFERRED EMBODIMENT OF THE INVENTION

Detailed embodiments of the instant invention are disclosed herein; however, it is to be understood that the disclosed embodiments are merely exemplary of the invention, which may be embodied in various forms. Therefore, specific functional and structural details disclosed herein are not to be interpreted as limiting, but merely as a basis for the claims and as a representation basis for teaching one skilled in the art to variously employ the present invention in virtually any appropriately detailed structure.

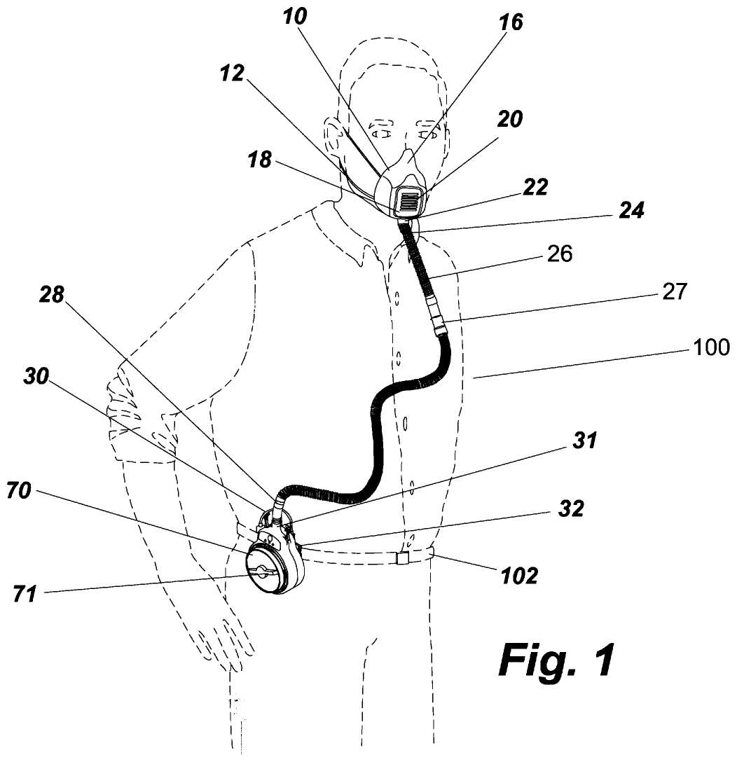

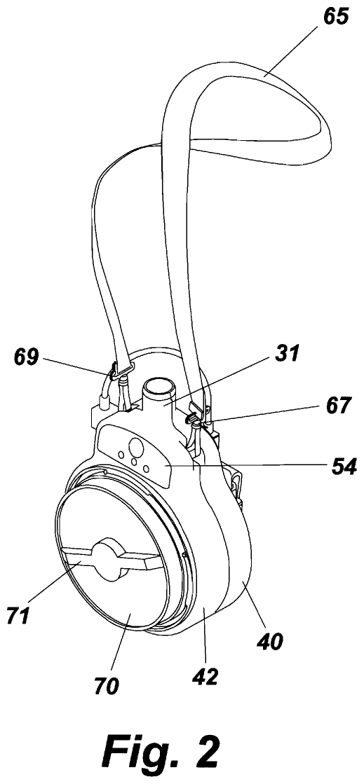

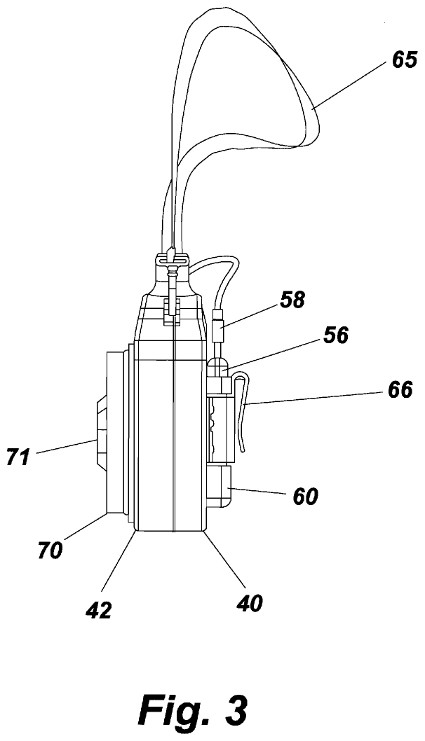

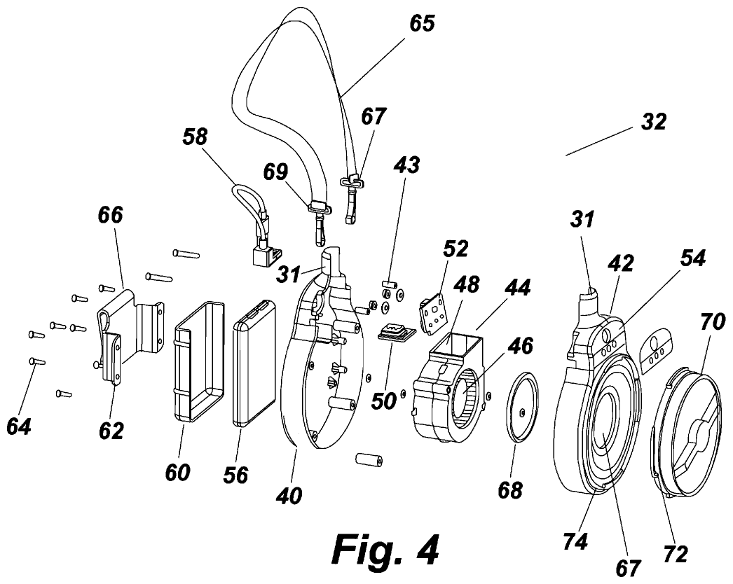

Referring to the FIGS. 1-5, illustrated is a portable rechargeable air handler having a HEPA or the like high quality air filter attached thereto, and having a face mask remotely attached to the air handler. The air purifier consists of a face mask 10 securable to an individual 100 by use of an elastic band 12 that can either wrap around the head of the individual or be placed over the ears of the individual. The face mask 10 is comprised of a clear plastic molded mask 16 having a filter 20. The filter to replaced every 2-4 months depending on the environmental conditions. A hose attachment port 22 secured to the proximal end 24 of an air hose 26 having a quick disconnect and swivel coupling 27. The coupling 27 allows the individual to move their head from left to right with minimal movement of the air hose 26. A distal end 28 of the air hose 26 is attached to a hose fitting 30 formed from a tubular shaped outlet 31 of a portable air handler 32.

The air handler 32 consists of a rear housing 40 and a front housing 42 which encompass a fan blade assembly 44; the blade assembly 44 having a plurality of blades 46 arranged in a circular array to provide air discharge through an upper section 48 fluidly coupled to the assembly outlet 31. The rear housing 40 is secured to the front housing 42 by fasteners 43. A programmable control board 52 is electrically coupled to a female USB connection operatively associated with the control board 52. In one embodiment, the airflow of the fan is 35 CFM, operating at 1200 rpm, drawing power of 1.1 Watt's at 17 dba. In another embodiment operation of the device using a two speed fan allow 9 plus hours operation on high and 15 plus hours on low speed. While 35 CFM is in one embodiment, an individual only needs about 1-2 CFM while at rest.

The programmable control board 52 is operable through a panel access point 54 with power provided by a lithium battery 56 through a USB charging cable 58. The battery 56 is slidably attached to the rear housing 40 by use of a battery pocket 60. A housing clip 62 is attached to the rear housing 40 by a plurality of fasteners; the housing clip 62 having a flexible sleeve 66, wherein the housing clip can be easily attached to an individual's belt 102. A flexible attachment strap 65 having end clips 67, 69 is securable to the air handler 32, wherein the air handler can be carried by hand or hoisted over a shoulder, similar to the carrying of a purse. An O-ring 68 provides a seal between the fan blade assembly 44 and the front housing 42, allowing air to be drawn through the aperture 67. A filter 70 is releasably securable to the front housing 42. Filter 70, preferably a HEPA filter, is for filtration of air drawn into the fan assembly 44. However, while a HEPA filter is the preferred embodiment, a filter equivalent to a KN95 may be used. The filter 70 may be a conventional KN95 face mask. Handle structure 71 is formed integral thereon and allows for ease of rotating the filter 70 between an attached and an unattached position without the use of tools. The filter 70 has tabs 72 which are inserted into a circular grooved recess 74 in the front housing 42 and turned in a clockwise direction to secure the tabs 72 within the grooved recess 74.

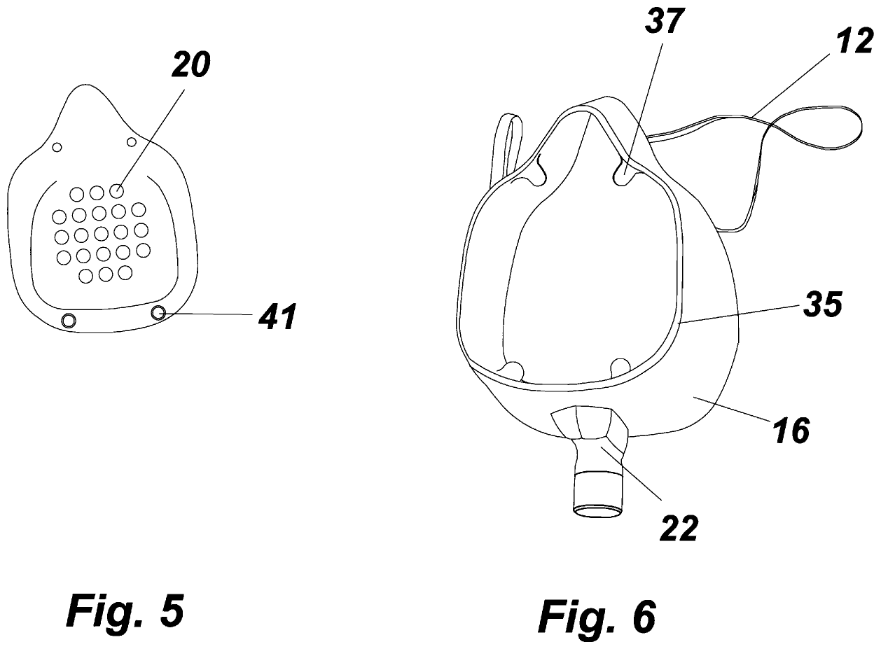

Referring to FIGS. 5 and 6, set forth is one embodiment of the face mask 16 with an opening 35 of a size wherein an individual could consume food or beverage without having to remove the mask 16 from the individual's face. In this embodiment the filter 20 is secured to the mask 16 by use of four magnets 37 positioned around the opening 35 for adhering to metal 41 formed in the filter housing 20. Positive flow of air continues to flow through the opening 35 providing a shielding effect of filtered intake air when filter 20 is removed. Wherein an individual maintains a level of protection while the filter 20 is removed to allows full frontal access. For instance, an individual may use a tissue to catch a sneeze, talk on the telephone or the like without having a filter 20 for temporary use, thereby maintaining a level of protection with the positive airflow.

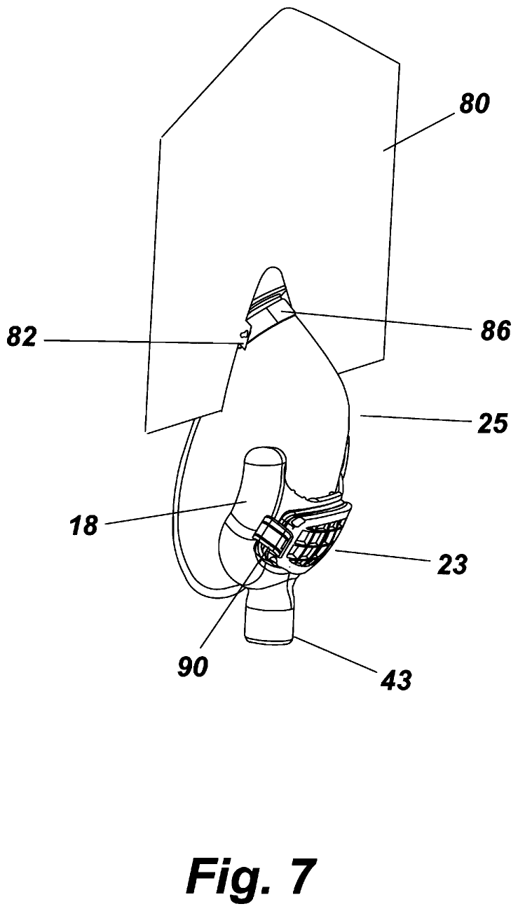

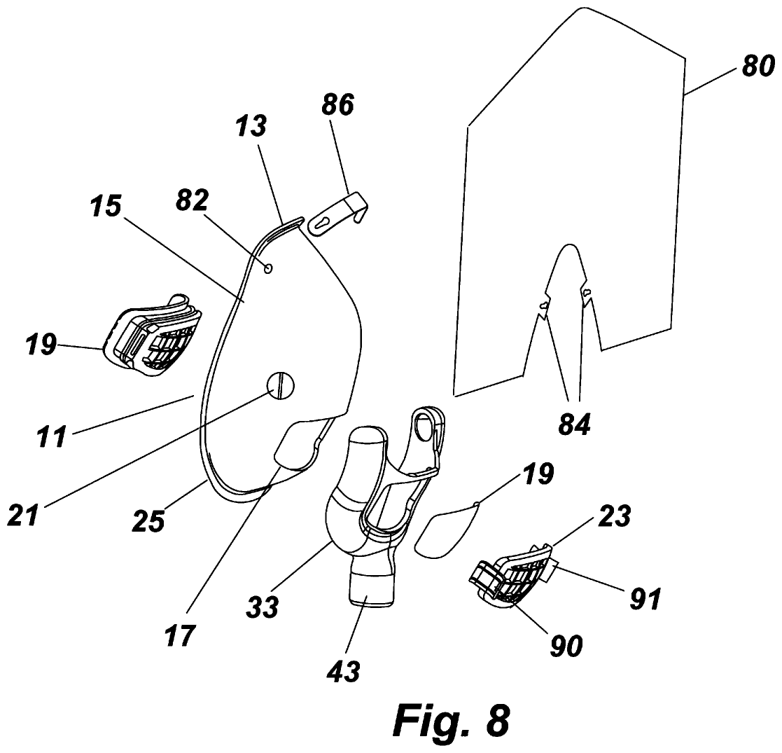

Referring to FIGS. 7 and 8, in this embodiment the face mask assembly is comprised of a clear plastic molded mask 25 having a mask duct 18 with a filter 19 inserted therein. A hose attachment port 43 is secured to the proximal end 24 of an air hose 26 having a quick disconnect coupling 27. A distal end 28 of the air hose 26 is attached by a hose fitting 30 to a tubular shaped outlet 31 of a portable air handler 32. The mask 25 has an opening 17 for receipt of a mask duct 33 which is is sealingly and rotatably securable to the mask 25 to encompass flow through holes 21. In this embodiment the mast duct 33 is receptive to a filter 19 which is removable allowing the individual to consume food or beverage as discussed in the previous embodiment. It is noted that the air handler 32 provides positive flow of air, wherein the individual's exhalation is conditioned as it is passed through the filter 19 so as to protect others from contaminates. In this embodiment, the filter 19 prevents contaminants from entering the face mask, but also conditions the air being expelled.

In another embodiment, the filter 19 in can be substituted with a high quality cotton or polypropylene material, particularly in vaccinated individuals. The filter 19 can be removed and left absent in certain situations, i.e. . . . high intensity outdoor activities such as riding a bike or jogging or where outdoor air quality is undesirable and unhealthy, and the individual will still be provided a constant supply of filtered, purified air through the positive air pressure from the air handler to the facemask.

A face shield film 80 can be attached to the face mask 25 by use of tabs 82 operatively associated with a shield receptacle 84 positioned near the nose bridge of the mask 25. A face shield tab 86 further couples to the tabs 82 to cause a formation of the film 80 to conform to the shape of an individual's face. As the air handler 32 provides a positive air flow, the filter 19 may be HEPA or N95, both of which are known to be excellent for filtration of viruses, but can quickly soil, making it hard to breathe through the filters. The positive air flow allows the air handler 32 to draw air into the unit and expel it through the face mask without additional effort by the user. The filter element retainer 23 has release levers 90, 91 for ease of retainer 23 removal, allowing access to the filter 19.

In summary, the portable air purifier is formed from a face mask 16 or 25 having a continuous sidewall 11 with an inner edge 13 and an outer edge 15. The inner edge 13 is constructed and arranged to cover an individual's nose and mouth. The inner edge 13 may include a foam seal, not shown, but preferably is formed for soft material that conforms to the individual's face. The first filter, or outtake filter 19 or 20, is removably attached to the face mask 16 or 25 by use of an exhaust port 19 secured to mast duct 18, and a filter element retainer 23. The filter 19 or 20 is capable of filtering 0.3-μm particles or smaller from passing through. An inlet aperture 17 is positioned in the face mask 16 or 25; a elastic band 12 is available for securing said face mask 16 or 25 to an individual; a air handler 32 consists of a rear housing 40 and a front housing 42 which encompass a fan blade assembly 44 having a plurality of blades 46 arranged in a circular array to provide discharge to said face mask inlet aperture 17; said fan blade assembly 44 is secured to the front housing 42 by fasteners and constructed and arranged to provide an airflow of 35 CFM, operating at 1200 rpm, drawing power of 1.1 Watt's at 17 dba. The fan is coupled to the face mask inlet opening aperture 17 by a flexible tube 26. A filter housing 70 is removably attached to the air handler 32, said intake filter capable of filtering 0.3-μm particles or smaller from passing through the filter. A power supply is provided by a lithium battery 56 coupled to the air handler through a control switch 52, said battery attached to said fan housing assembly by use of a battery pocket 60; whereby the air handler 32 draws air through said intake filter 70 and passes the filtered air to said face mask with a positive flow for receipt by a face mask wearer, and exhausted through said filter 19 or 20, wherein incoming air is filtered for the safety of the face mask wearer and air expelled by the face mask wearer is filtered by said filter 19 or 20 for the safety of others.

The term “coupled” is defined as connected, although not necessarily directly, and not necessarily mechanically. The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

All patents and publications mentioned in this specification are indicative of the levels of those skilled in the art to which the invention pertains. It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A portable air purifier:
a face mask base formed from a continuous sidewall defined by an inner edge and an outer edge, said inner edge constructed and arranged to cover an individual's nose and mouth, said outer edge forming a opening providing access to the individuals' nose and mouth, said continuous sidewall including an inlet aperture;
an outtake filter removably attached to said opening;
a air handler consisting of a rear housing securable to a front housing forming a chamber for placement of a fan blade assembly having a plurality of blades arranged in a circular array to provide airflow through said inlet aperture, said fan blade assembly is secured to the chamber providing said 35 CFM airflow;
an intake filter removably attached to said air handler;
a power supply provided by a lithium battery attached to said air handler by an external pocket and electrically coupled to said air handler through a control switch;
whereby the airflow is drawn through said intake filter and directed to said face mask and exhausted through said outtake filter, wherein said face mask opening is constructed and arranged to allow an individual to continue to receive airflow allowing the individual to consume food, beverages or the like while the face mask base covers the individual's nose and mouth.

2. The portable face mask according to claim 1 wherein said intake filter and outtake filter prevent particles 0.3-μm or smaller from passing through the filter.

3. The portable face mask according to claim 2 wherein said intake and outtake filter are HEPA filters.

4. The portable face mask according to claim 2 wherein said intake and outtake filters are N95 filters.

5. The portable face mask according to claim 2 wherein said intake filter is selected from the group consisting of high quality cotton or polypropylene material.

6. The portable face mask according to claim 1 wherein said housing includes a clip for securing said air handler to an individual's waist belt.

7. The portable face mask according to claim 1 wherein said battery is rechargeable while positioned in said battery pocket.

8. The portable face mask according to claim 1 wherein said battery is securable to a USB connector to allow static or dynamic use.

9. The portable face mask according to claim 1 including a face shield film securable to said face mask.

10. The portable face mask according to claim 1 wherein said fan blade assembly operates at about 1200 rpm drawing less than 1.1 W at less than 17 dba.

11. The portable face mask according to claim 1 including at least one elastic strap for securing said face mask to an individual.
